(19) United States
(12) Patent Application Publication
(10) Pub. No.: US 2003/0144362 A1
(43) Pub. Date: Jul. 31, 2003

(54) High viscosity antibacterials for cannulae

(76) Inventors: David S. Utterberg, Seattle, WA (US); Fred G. Swindler, Bellevue, WA (US); Garrettson Ellis, Northfield, IL (US)

Correspondence Address: SEYFARTH SHAW, 55 EAST MONROE STREET, SUITE 4200, CHICAGO, IL, 60603-5803, US

(21) Appl. No.: 10/056,045

(22) Filed: Jan. 28, 2002

(51) Int. Cl. A61K 31/045; A61K 9/14; B05D 3/00

(52) U.S. Cl. 514/724; 427/2.28

(57) ABSTRACT

An antibacterial fluid may be applied to a tubular medical cannula for access to a patient. The fluid comprises a metabolizable antibacterial formulation having a viscosity of at least about 5,000 cp. The cannula may then be inserted into the patient with an increased lubricity for a reduction of pain, while at the same time, unlike silicones, materials do not readily accumulate in the patient. The tubular medical cannula may be a rigid, hollow needle, sharp or blunt, a spike, or a flexible catheter. Also, the viscous antibacterial fluid may be used to lock a catheter or other cannula while implanted in the patient, for storage purposes.

### BACKGROUND OF THE INVENTION

In the area of hemodialysis and other forms of therapy which require repeated access to the vascular system of a patient, the problem of vascular access remains significant, in large measure because of the problems with infection, and with clotting of blood in vascular access catheters.

One approach to the technical problem of effective, repeated vascular access involves the use of an implantable artificial port which is positioned under the skin of the patient. Then, a needle passes through the skin of the patient into the port to provide the vascular access.

Examples of such technology are illustrated by Finch et al. U.S. Pat. No. 5,562,617, Enegren et al. U.S. Pat. No. 4,955,861, and PCT International Publications WO97/47338; WO98/31416; and WO99/03527.

Needles which are used for access to the body may connect with such implanted ports, or they may connect with an arteriovenous fistula, or grafts, as is common in the art of hemodialysis and other extracorporeal blood therapies, or may cannulate any other body lumen or tissue, as in an intramuscular injection.

Such needles desirably have a silicone lubricant on their exterior surface to serve as a lubricant. This can significantly reduce the pain of the needle stick. However, silicone is not well metabolized, and is retained by the body. Thus, even though only tiny amounts of silicone enter into the patient with each needle stick, the amount of silicone can accumulate especially in patients who have lost their kidney function. Thus, there is a dilemma, in that to reduce patient pain it would be desirable to use a bit more silicone on the needle surface, while to reduce the accumulation of silicone in the patient, it is desirable to use little or no silicone.

Furthermore, silicone is not antibacterial in nature, i.e. it is neither bacteriostatic nor bactericidal.

Other attempts have been made to provide lubricating coating to needles. One of them, known as Spire coating is lubricating only after they have been hydrated. This takes a little time, and thus they are more useful for catheters which enter the body through previously made incisions than they are for cutting needles or other rigid cannulae.

Furthermore, needles may pass through the skin repeatedly through the same track (called a cannula or needle “tract” herein) so that they do not break through new tissue as they pass through the skin to engage an implanted port. This needle tract, which represents a passageway through which fluids may flow and bacteria may pass, is desirably flushed in reverse matter from the inner end of the needle tract to the outer end and through the skin, to remove bacteria which may have been drawn in by needle penetration or the like. However, current antibacterial flushing solutions have the additional disadvantage that they require time and expense to administer (e.g. by syringe and needle) and the effluent may dribble down the skin of the patient after coming out of the needle tract in an inconvenient and undesirable manner, since the dialysis position taken by patients is frequently semi-upright.

Further, typical topical disinfectants like isopropyl alcohol used in skin prep scrubs tend to evaporate before they can completely kill the bacteria they initially contact. It would be advantageous if a means to retard the evaporative process for a volatile skin prep disinfectant were available.

It is also desirable to have anti-bacterial fluid surrounding the needle site during the procedure when the needle or other percutaneous device is implanted through the skin and communicating with an implanted port, so as to have an active disinfecting and/or physical barrier to block organisms from entering the annular tunnel between the cannula and the needle tract. Such antibacterial fluids generally need to be held within a gauze pad to prevent draining away from the needle or cannula tract site. However, the gauze provides increased wicking surface area, causing the antibacterial fluid to evaporate even more quickly than without the gauze. Evaporation stops the antibacterial action at the entrance to the cannula tract or the “tunnel.” Thus, it is necessary to be rather vigilant, repeatedly adding antibacterial fluid to the area around the outer entrance of the tunnel or needle tract.

Also, such needle tracts may be accidentally innoculated with bacteria due to bacteria alighting on an exposed needle, or otherwise being dragged in by the advancement of the needle through the needle tract from surrounding contaminated tissue or air. Conventional antibacterial fluids used to flush the needle tract or tunnel are of low viscosity, and thus migrate out of the tract and evaporate in fairly short order, causing the area between the needle and the needle tract to become a place where bacteria can grow. Additionally, conventional disinfectants such as alcohols are typically volatile at low temperatures, and thus evaporate quickly from their site of application before they have time to kill all microorganisms present.

Furthermore, there is a need to “lock” implanted catheters, by which is meant that an antithrombogenic solution such as heparin solution is placed into a catheter lumen which is implanted in the body, to suppress clotting as the blood migrates into the lumen of the catheter when it is not in use, such as between dialysis procedures. In the absence of such a catheter lock, substantial quantities of blood may migrate into the lumen of the catheter and clot there, rendering the implanted catheter useless.

However, because of the low viscosity of the typical antithrombogenic formulations containing heparin (and optionally antibacterial components such as alcohol or citric acid) the catheter lock solution diffuses away, and is replaced to a certain extent by blood during the period between dialyses, which may be on the order of 48 to 72 hours. Also, as the catheter lock solution diffuses slowly into the patient, its ingredients such as heparin, alcohol, citrate, citric acid, etc. get into the patient. This may result in certain toxic effects over the long run, since the catheter lock procedure is being used on a chronic basis between each dialysis procedure. For example, while isopropyl alcohol is a good antibacterial ingredient and is metabolizable, a study from Germany reports that toxic symptoms can arise with a daily dose exceeding only 500 mg of isopropyl alcohol.

Also, even conventional needles can be contaminated before use by exposure to air, for example when a particle of dust lands on the needle. This can be a source of unsterility when the needle enters the patient, or a needle or spike enters a sterile Y site, injection site or ampule.

The technical problems described above are reduced by the invention of this application, as described below.

### DESCRIPTION OF THE INVENTION

In accordance with one aspect of this invention, an antibacterial (antiseptic) fluid or gel may be applied to a tubular medical cannula (that is, a needle, catheter, or tubular spike) for access to a patient or medical device communicating with a patient, where the fluid or gel comprises an antibacterial formulation having an elevated viscosity over aqueous solutions such as normal saline solution and povidone iodine. Preferably, the elevated viscosity may be about 5,000 to 80,000 centipoise (cp) when measured, although a gel may be self-supporting, essentially without flow characteristics until it is disturbed. The viscosities stated herein are as measured by a Brookfield viscometer at 22° C. with an RV6 spindle at ten r.p.m. The cannula may be inserted into the patient. The word “antibacterial” implies antiseptic effect against fungi also, and other microbes such as protozoa.

The antibacterial fluid or gel may be applied by the manufacturer, the cannula being packaged to avoid evaporation. Otherwise, the fluid or gel may be applied by a nurse at the site of use by dipping the cannula, into it or passing it through the fluid or gel on the skin, for example.

The antibacterial fluid or gel may be placed on the outer wall of the cannula to serve as a lubricant for a sharp ended needle or a blunt ended cannula, for access to an implanted port, or alternatively to facilitate direct access by the cannula to a fistula or other blood vessel of the patient. Preferably, the fluid or gel (hereafter generally called “fluid”) has a lubricating capability to reduce the friction of the cannula which is advancing into the patient, when compared with the same cannula advancement without the fluid. Generally, this lubricating effect is found spontaneously with increased viscosity of the fluid used in this invention. Preferably, the viscosity of the antibacterial fluid of this invention may be 10,000 to 50,000 cp. Also, the fluid evaporates less quickly, retaining antibacterial ingredients such as alcohols, for improved antibacterial effect.

The fluid of this invention may be placed on the cannula outer wall in an amount which is sufficient to cause some of the fluid to be wiped from the cannula upon said inserting of the cannula into the patient, so that a ring portion of the fluid visibly resides adjacent to the skin of the patient. This provides a typically annular, antibacterial barrier at the outer end of a cannula tract that evaporates slowly, to suppress the entering and growth of bacteria and other microorganisms into the cannula tract. Alternatively, a small (such as a 2 cm. diameter) pool of the fluid may be placed on the skin at the cannula entry site, and the dry cannula may be passed into the skin through the pool. Thus, some of the fluid may adhere to the cannula and pass into the needle tract, for antibacterial action there, while the pool provides an antibacterial seal at the needle entrance. The high viscosity fluid reduces the evaporation of alcohols and other antibacterial agents in it, greatly prolonging the antibacterial action.

Typically, the antibacterial fluid of this invention comprises a low viscosity antibacterial agent mixed with a viscosity increasing agent. Examples of antibacterial agents which may be used comprise alcohols, chlorhexidine, Chlorpactin, iodine, tauroline, citric acid, and soluble citric acid salts, particularly sodium citrate, optionally mixed with water.

Examples of viscosity increasing agents comprise Carbopol, starch, methylcellulose, carboxypolymethylene, carboxymethyl cellulose, hydroxypropylcellulose, or the like, preferably a material such as starch which can clear out of the body of the patient by metabolization or excretion in the quantities used, so that the material does not accumulate in the body. This property is defined herein by the phrase “body clearing”. Carbopol is a cross-linked polyacrylic acid based polymer sold by Noveon, Inc. It is preferably neutralized to about pH 7 with a base material such as tetrahydroxypropyl ethylene diamine, triethanolamine, or sodium hydroxide. Derivatives of starch may also be used, such as hydroxyethylstarch, hydroxypropylstarch, or starch having bonded organic acid ester groups, to improve compatibility with antibacterial agents such as alcohols, for example, ethanol or isopropanol. Such ester groups may be the reaction product of two to twelve carbon organic acids with the starch, for example. Also, the elevated viscosity antiseptic fluid may be created by the use of a fat emulsion, or other dispersions in water/alcohol of glycerol mono or di esters of fatty acids, or fatty acid esters of other polyols such as sugars having one or more bonded fatty acid groups per molecule. Analogous compounds with ether linkages may also be used.

Also, other materials such as alginic acid, with or without calcium citrate may be used, or polyvinyl alcohol, with or without borax, povidone, polyethylene glycol alginate, sodium alginate, and/or tragacanth.

These ingredients may be admixed to form the fluid of this invention at any desired elevated viscosity, for the purpose of achieving the advantages of this invention by reducing the disadvantages discussed above, while also providing needle lubrication when desired. If desired, the fluid of this invention may also contain an effective amount of an antithrombogenic agent such as heparin, and a diluent such as water, along with other desired ingredients.

Alternatively, or additionally, the fluid of this invention may be applied to the lumen of a cannula such as a catheter, to provide a lock that restricts the flowing of body fluids into the cannula. Also, the fluid of this invention may be used with any cannula, spike, catheter, or the like for any purpose, to provide a retentive, self-sterilizing characteristic to the product.

In one embodiment, the formulation of this invention may comprise a mixture of isopropyl alcohol and neutralized Carbopol, with other optional ingredients being present such as water, antithrombogenic agents such as heparin, and the like. Preferably, about 0.4 to 2 weight percent of Carbopol is present. Citric acid may also be present as an antibacterial agent, either with or as a substitute for another anti-bacterial agent such as isopropyl alcohol or ethanol.

In another embodiment, a gel of isopropyl alcohol, optionally with up to about 30 weight percent water, may be formed with 2.2 weight percent hydroxypropylcellulose, to form a high viscosity antibacterial agent of this invention.

The antibacterial, viscous fluid of this invention may be provided to the user in an inexpensive squeeze-delivery container, to avoid the need for a syringe or other more expensive delivery system. A squeeze-delivery container may be a one piece, blow molded container in which the contents are administered by simple manual squeezing of the fingers. Specifically, the squeeze-delivery container which holds the antibacterial fluid of this invention may carry a male luer typically having an inner diameter at its tip of least about 2 millimeters. One may attach the male luer of the container to a female luer of a rigid cannula or catheter, which may be emplaced in the body of a patient. One then squeezes the container for a simple transfer of the antibacterial formulation into the rigid cannula or catheter.

Further in accordance with this invention, one may flush a preferably metabolizable, antibacterial fluid through a cannula tract which extends through the skin of a patient and inwardly therefrom. The method comprises the steps of inserting a cannula into the cannula tract; and passing the fluid through the cannula to exit the cannula at an inner portion of the tract, and to cause the fluid to flow outwardly through the tract outside of the cannula so that some of the fluid exits around the cannula through the skin, where some of it is retained. The antibacterial fluid preferably has a viscosity of about 10,000-30,000 cp, and it may be a formulation similar to that previously described. The cannula tract may communicate its inner end with an implanted, artificial port, which communicates with a body lumen of a patient.

Furthermore by this invention, one may place a preferably metabolizable fluid into a lumen of a catheter installed in a patient, typically a permanently implanted catheter, to “lock” the catheter, reducing the migration of body fluids into the catheter lumen while the catheter is not in use, to thus avoid clotting as the catheter resides in the patient. The fluid preferably has a viscosity of about 10,000-50,000 cp, and may be a fluid as previously described. Such fluids may comprise an antibacterial agent and/or an antithrombogenic agent.

This “lock” can be achieved because of the increased viscosity of the fluid in accordance with this invention, which thus physically resists removal from the lumen of the catheter and replacement by blood while residing in the body between uses of the catheter. Also, as previously taught, there may be present an antibacterial agent and/or an antithrombogenic agent. For example, a gelled heparin solution at a suitable concentration may be used, exhibiting the elevated viscosity on testing of preferably about 5,000-80,000 cp, when measured, so that any blood that does enter into the lumen is going to encounter conditions where clotting is suppressed because of the presence of heparin, and microbial growth may be suppressed when an antibacterial agent is present.

Also, by this invention, a preferably body clearing, antibacterial fluid described above can be used to coat hypodermic needles, spikes or the like to reduce needle contamination, since the needle or spike comprise an actively disinfecting surface film. Simultaneously, the fluid material of this invention may be used as a desirable needle lubricant, but providing active sterility so that dust particles that land on the needle when the needle is exposed to the air, or other contamination, tend to be sterilized so that the contamination does not spread to the patient, or to a sterile Y site, ampule, or the like.

Additionally, the formulations of this invention maybe squeezed out onto the skin, especially when gel-like in consistency, preferably at a viscosity of about 20,000 to 50,000 cp, to form a little sterilizing pool on the skin. The gel retards the evaporation of the disinfecting medium, thus giving greater “contact time” of said medium with any infecting agent it encounters on the skin. Additionally, it retards the movement of the pool by gravity or patient movement. Then, a needle may pass through the viscous material of this invention, to provide further assurance of sterile entry of the needle and subsequent protection along the needle and at the skin entry point with less evaporation of antiseptic than with current techniques. This may be used with fistula needles in hemodialysis and the like, with good needle lubrication being provided for reduced pain,

### DESCRIPTION OF DRAWINGS

Referring to the drawings,

FIG. 1 is a vertical section of a tubular medical cannula, shown to be penetrating the skin of the patient and connecting with an implanted artificial port, which is shown in schematic form.

FIG. 2 is an elevational view of a catheter which is implanted to extend through the skin of the patient and to connect with an implanted artificial port, with the catheter being releasably connected with a container of the antibacterial fluid of this invention.

FIG. 3 is a schematic view of separated components of a medical kit, the components being for practicing methods of this invention.

### DESCRIPTION OF SPECIFIC EMBODIMENTS

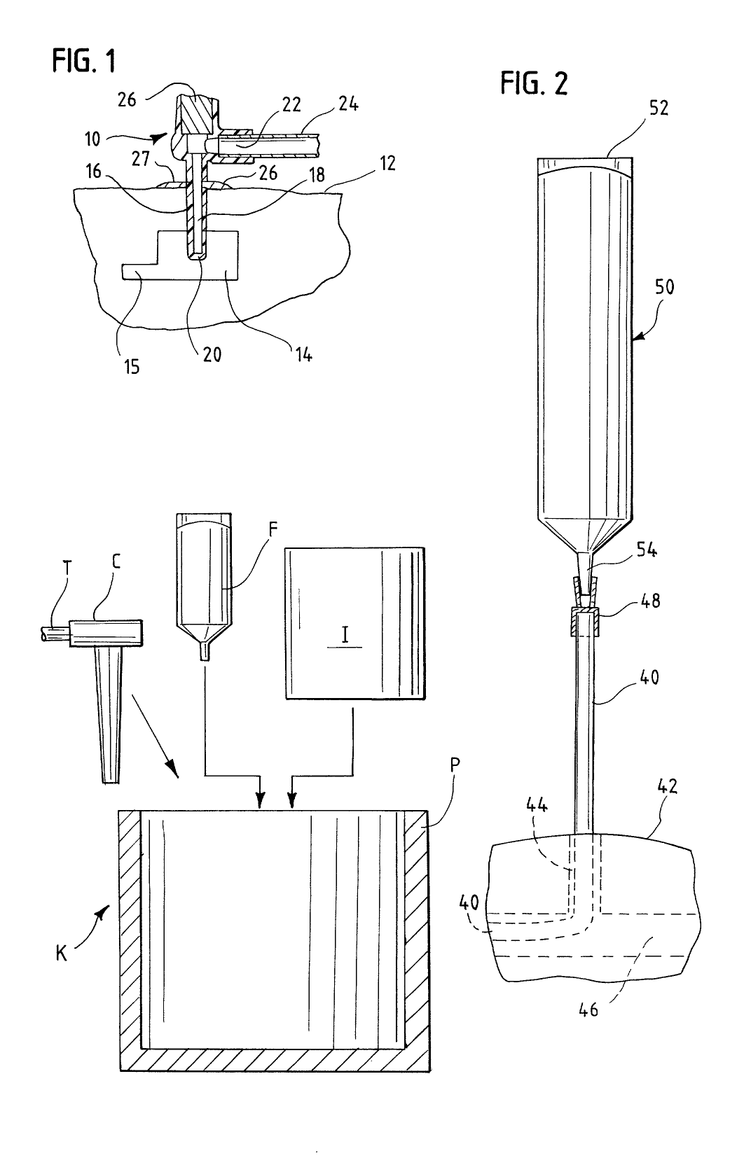

Referring to FIG. 1, an angled cannula 10 is shown to be penetrating the skin 12 of a patient, to extend along a cannula or needle tract 16 through tissue of the patient to enter into sealing, flow communication with a port 14, implanted within the tissue of the patient under the skin 12. Broadly speaking, the technique is similar to that discussed in the PCT publications WO98/31416 and WO99/03527, as cited above. Conduit 15 is connected to a blood vessel of the patient. A known valve is present to control flow through conduit 15.

Cannula set 10 carries a rigid cannula 18 which may either have a sharp tip or a blunt tip 20, to provide communication through the skin 12 between the implanted port 14 and a flow conduit 22, which may comprise a conduit through cannula member 10 as shown, which conduit may also extend into the lumen of connected, flexible tubing 24. A suitable resealable plug 26 may be provided, carrying a preformed slit if desired, to provide needle access to the flow conduit through resealable plug 26, as previously disclosed in Utterberg et al. U.S. Pat. No. 6,267,750, entitled Tapered Intravenous Cannula. As disclosed there, cannula 18 may also be tapered and blunt, if desired.

In accordance with this invention, cannula 18 may be inserted into cannula or needle tract 16, which may be a preformed tract created by previous cannula penetrations so that the preferably blunt cannula 18 does not cut through tissue which has not been previously cut by prior penetrations of cannula needles, to facilitate the penetration of cannula 18 into needle tract 16 without pain.

An antibacterial fluid having a lubricating capacity may be provided to the outer surface of cannula 18, to reduce the friction of cannula 18 advancing into the patient. For example this fluid has a viscosity of about 25,000 cp. Preferably, this antibacterial fluid is an aqueous solution of about 50 to 90 weight percent of ethyl alcohol or isopropyl alcohol, from zero to 10 weight percent of dissolved citric acid, and sufficient viscosity increasing agent, particularly neutralized Carbopol, hydroxypropylcellulose, or a starch derivative, to provide the desired viscosity to the aqueous solution. Typically, about 10 to 40 weight percent of water will be present. Typically, from 0.4 to 0.7 weight percent of Carbopol may be used, or from 2 to 4 weight percent of hydroxypropylcellulose.

For example, specific formulations may comprise an aqueous isopropyl alcohol solution (70% alcohol and 30% water) containing 0.5 weight percent of neutralized Carbopol, or 2.2 weight percent of hydroxypropylcellulose, to provide a viscous, gel-like material.

A sufficient amount of the fluid of this invention may be placed on the outer wall of cannula 18 so that, as cannula 18 advances through cannula tract 16, some of the fluid is wiped from the cannula and visibly resides in the annular junction 26 between the cannula 18 and the skin 12, to serve as an antiseptic reservoir at the outer end of needle tract 16, thus protecting the tubular opening defined by needle tract 16 between cannula 18 and the wall of needle tract 16. Alternatively, one may place a small portion of the viscous, gel-like fluid 27 on the skin over needle tract 16, passing cannula 18 through it into needle tract 16. Thus the pool of fluid 27 forms a continuing antibacterial seal that holds its antiseptic such as alcohol with less evaporation, for better antibacterial action. Hydroxypropyl cellulose serves well to provide a suitable, stable, gel-like emulsion.

If desired, an effective amount of an antithrombogenic agent such as heparin may also be added to the antibacterial fluid of this invention.

The typical purpose of the connection of cannula member 10 and implanted port 14 is to provide access for extracorporeal blood transport between the vascular system of the patient and an extracorporeal blood processing device such as a hemodialyzer. Two of such connections of the type as shown in FIG. 1 may be typically used in a hemodialysis process, with the blood passing into cannula 18 from port 14, which connects with a vein of the patient. The blood then passes through tubing 24 to a dialyzer or other blood treatment device, and then is correspondingly returned through another, similar connection.

Alternatively, about 1 to 4 weight percent of ethylcellulose, hydroxyethylstarch, or hydroxypropylstarch may be used as the viscosity increasing agent.

Further in accordance with this invention, after cannula 18 has been inserted into needle or cannula tract 16 as shown in FIG. 1, extending through the skin of the patient, preferably a metabolizable, antibacterial fluid in accordance with this preferred embodiment is passed through cannula 18 inwardly, to exit the cannula at end 20. As is known, implantable port 14 may have a valve so that the antibacterial fluid from cannula 18 cannot pass further into port 14, but rather, the fluid then flows outwardly through tract 16, outside of cannula 18, to flush cannula tract 16 in a known manner (but for the composition of the antibacterial fluid of this invention,) taking with it bacteria and other contamination to reduce infection. By way of advantage, the fluid has an increased viscosity of at least 5,000 or 10,000 cp, and preferably 20,000-30,000 cp, so as to be able to flush cannula tract 16, while being immobile enough through its elevated viscosity to resist migration out of the tract 16, and away from annularjunction 26, when positive flushing is not taking place. Thus, better antibacterial effect may be provided while cannula 18 resides in cannula tract 16.

It also may be desirable to allow the fluid of this invention to reside in the lumen of cannula 18 to serve as a “lock”, i.e. a protection against the migration of stagnant blood into the cannula while it is not being used, to prevent against clotting of blood and bacteria build up within the cannula, and to reduce chances of forming a biofilm that can reduce flow through cannula 18.

If desired, the antibacterial fluid of this invention may be administered by a syringe or other container through resealable needle access plug 26. Also, when desired, such antibacterial fluid can be removed from cannula 18 in a similar manner, when it is undesirable to commingle the entire aliquot of antibacterial fluid with blood or other fluid normally transported through the system during use. The viscous fluid is better retained in a cannula or catheter, particularly at viscosities of 10,000 cp or higher.

The antibacterial fluid used is also preferably antimicrobial in nature, to prevent the growth of bacteria, fungi, and other microorganisms.

Referring to FIG. 2, another type of use of the antibacterial fluid of this invention is shown. An implanted catheter 40 is shown extending inwardly through the skin 42 of a patient, passing through a tissue tunnel 44 and being sutured into communication with a vein 46 of the patient for obtaining blood access to the patient, for extracorporeal blood processing such as hemodialysis. Often, two such implanted catheters are provided to a patient.

Catheter 40 terminates in a female luer connector 48. By this invention, a squeeze-delivery container 50, containing the antibacterial fluid of this invention, is provided. Container 50 may comprise a blow molded container, or a length of flexible tubing sealed at its upper end 52, and carrying an integral male luer connector 54 at its lower end, capable of releasable sealing engagement with female luer connector 48. Preferably, male luer 54 has a lumen with an inner diameter of at least 2 mm.

Thus, after attachment of container 50, which holds the viscous fluid of this invention, one may squeeze container 50 between uses of catheter 40 to substantially fill catheter 40 with the viscous fluid of this invention, thus providing a “catheter lock”. The fluid viscosity may preferably be about 30,000 to 40,000 cp. This lock suppresses the migration of blood into catheter 40, where the blood can clot and block flow in the catheter. Also, microorganism growth within catheter 40 is reduced, as well as the formation of biofilms, which can eliminate catheter usefulness by blocking blood diffusion flow into the catheter. Because of the increased viscosity of the antibacterial fluid of this invention, it is more effective as a catheter lock than known solutions, lasting for several days while reducing the migration of blood into the catheter lumen during storage.

When it is desired to open the catheter again for extracorporeal blood flow, the fluid of this invention filling catheter 40 during the catheter lock period is optionally removed by a syringe or the like through connector 48, so that most of the antibacterial fluid is not mixed with blood of the patient. However, those amounts of the antibacterial fluid which are mixed can readily be cleared by the body with proper selection of ingredients in accordance with this invention.

Here also it may be desirable to incorporate an antithrombogenic agent such as heparin into the antibacterial fluid in an effective concentration, to suppress the clotting of any blood that does find its way into catheter 40 during the catheter lock period.

Referring to FIG. 3, a kit is shown in exploded condition for practicing the various methods of this invention. A set comprising a length of tubing T, connected to a tubular medical cannula C for access to the patient, is provided. Alternatively, cannula C may comprise a catheter for connection with the blood supply of a patient, if desired. Alternatively, element C and connected tubing T may be eliminated from kit K.

Kit K also contains a fluid container F of the fluid of this invention, for application either to a catheter or a rigid cannula. Packaging unit P is also provided to contain the various elements of the kit, the packaging unit P being a sealable envelope, typically capable of gas sterilization, or a tray with a porous cover having similar sterilization capability, or the like.

Instructions I are also included, providing instructions on the use of the fluid F of this invention in conjunction with cannula or catheter C in accordance with any of the previously described methods for applying antibacterial fluid to a medical cannula such as a rigid needle, a flexible catheter, or the like, as previously described.

Preferably, because of increased viscosity, the antibacterial fluid of this invention significantly reduces the friction of a needle or other cannula as it is advanced into the patient, typically a catheter, a fistula needle, or a cannula entering through a cannula or needle tract. The fluids of this invention are instantly lubricious, and do not require a hydration step, as is the case for some catheter lubricants. There can be antibacterial characteristics, which provide significant advantage over such hydratable materials and silicones. The preferred fluids of this invention also are retained more persistently on the skin in the vicinity of a catheter or rigid cannula within the patient because of the increased viscosity, resulting in the significant advantage of better antibacterial effect. Also, they are less likely to evaporate or dribble away from the needle or cannula tract along the skin. The fluid of this invention may coat the interior walls of a catheter, with the bulk fluid being removed. The increased viscosity of the fluid can create such a coating, to durably act as an antimicrobial agent without the presence of the bulk fluid filling the catheter or other cannula.

Medical needles of anytype may have their surfaces liberally applied as described above with the viscous, antibacterial fluid of this invention for increased comfort to a patient, while the needle retains a self-sterilizing characteristic as the needle is inserted, with less concern about the accumulation of materials from the fluid in the patient over the long term. Fistula needles for dialysis may be so coated, retaining better sterility as they are exposed to the air during the priming process.

The above has been offered for illustrative purposes only, and is not intended to limit the scope of the invention of this application, which is as defined in the claims below.

That which is claimed is:

1. The method which comprises: applying an antibacterial fluid to a tubular medical cannula for access to a patient, said fluid comprising an antibacterial formulation having a viscosity of about 5,000 to 80,000 cp, and inserting the cannula into the patient or a medical device communicating with the patient.

2. The method of claim 1 in which said fluid is placed on an outer wall of the cannula, and said fluid has a lubricating capability to reduce the friction of the cannula advancing into the patient, when compared with the same cannula advancement without said fluid.

3. The method of claim 2 in which said fluid is placed on the cannula outer wall in an amount sufficient to cause some of said fluid to be wiped from the cannula upon said inserting of said cannula, to visibly reside adjacent to the skin of the patient.

4. The method of claim 1 in which said cannula is passed through a pool of said fluid residing on the skin of the patient.

5. The method of claim 1 in which said fluid comprises an antibacterial agent, mixed with a body-clearing, viscosity increasing agent.

6. The method of claim 1 in which said antibacterial agent comprises isopropyl alcohol.

7. The method of claim 1 in which said viscosity increasing agent comprises starch.

8. The method of claim 1 in which said fluid is applied to the interior of the cannula.

9. A kit which comprises a tubular medical cannula, a supply of antibacterial fluid having a viscosity of about 5,000 to 80,000 cp, and instructions for practicing the method of claim 1.

10. An antibacterial formulation which comprises a relatively low viscosity antibacterial agent mixed with sufficient viscosity increasing agent to provide a viscosity of about 5,000 to 80,000 cp to the formulation.

11. The formulation of claim 10 in which said low viscosity antibacterial agent is selected from the group consisting of alcohols, chlorhexidine, chlorpactin, iodine, tauroline, citric acid, and soluble citric acid salts such as sodium citrate.

12. The formulation of claim 10 which comprises an effective amount of an antithrombogenic agent such as heparin.

13. The formulation of claim 10 in which said antibacterial agent comprises at least one of ethanol, isopropanol, and citric acid.

14. The formulation of claim 10 in which said viscosity increasing agent comprises hydroxypropylcellulose.

15. The formulation of claim 10 in which said viscosity is from 20,000 to 30,000 cp

16. The formulation of claim 10 which comprises a mixture of isopropyl alcohol and about 2 to four weight percent of hydroxypropylcellulose.

17. A squeeze-delivery container which contains the formulation of claim 10.

18. The container of claim 17 in which said container has a delivery port which comprises a male luer with lumen having an inner diameter of at least 2 mm.

19. A cannula, carried by a hub and having a lumen at least partially filled with the formulation of claim 10.

20. The formulation of claim 10 which is body-clearing.

21. The formulation of claim 20 in which said viscosity increasing agent comprises a starch.

22. The method which comprises: attaching the male luer of the container of claim 17 to a female luer of a catheter emplaced in the body of a patient, and squeezing said container to transfer said antibacterial formulation into said catheter.

23. The method which comprises placing an antibacterial fluid in a cannula tract extending through the skin of a patient and inwardly therefrom, said method comprising; inserting a cannula coated with said fluid into said cannula tract; and passing flushing fluid through the cannula to exit said cannula at an inner portion of said tract and to cause said flushing fluid to flow outwardly through said tract so that some of said fluid exits around the cannula through the skin; said antibacterial fluid having a viscosity of about 5,000 to 80,000 cp.

24. The method of claim 23 in which said antibacterial fluid has a viscosity of 10,000 to 30,000 cp, and comprises a relatively low viscosity antibacterial agent plus a viscosity increasing agent.

25. The method of claim 23 in which said antibacterial agent is isopropyl alcohol.

26. The method of claim 23 in which said viscosity increasing agent comprises hydroxypropylcellulose or a starch.

27. The method of claim 23 in which said cannula tract communicates at its inner and with an artificial port which communicates with a body lumen of a patient.

28. A kit which comprises a cannula, a supply of antibacterial fluid having a viscosity of about 5,000 to 80,000 cp, and instructions for practicing the method of claim 23.

29. The method which comprises: placing a fluid into a lumen of a catheter installed in a patient to “lock” the catheter, to reduce the flow of body fluids into the catheter lumen as the catheter resides in the patient, said fluid having a viscosity of about 5,000 to 80,000 cp.

30. The method of claim 29 in which said fluid comprises an antibacterial agent.

31. The method of claim 29 in which said fluid comprises an antithrombogenic agent.

32. A kit which comprises a supply of fluid to “lock” an installed catheter, said fluid having a viscosity of about 5,000 to 80,000 cp, and instructions for practicing the method of claim 29.

33. The method which comprises placing a fluid on the outer surface of a medical cannula, said fluid comprising an antibacterial agent and having a viscosity of about 5,000 to 80,000 cp, and thereafter inserting the cannula through the skin of the patient or into a sterile receptacle.

34. The method which comprises placing a portion of a fluid, which comprises an antibacterial agent and has a viscosity of about 5,000 cp to 80,000 cp, on the skin of a patient to form a fluid layer on the skin, and thereafter passing a medical cannula through the fluid layer on the skin and through the skin of the patient.

35. The method of claim 34 in which said medical cannula is carried by a hub and connects with flexible tubing, said cannula extending transversely to the axis of said flexible tubing adjacent to said hub.

36. An aqueous solution of an antithrombogenic agent, present in a concentration effective to suppress blood clotting, said solution also comprising a viscosity increasing agent and having a viscosity of 5,000 to 80,000 cp.

37. The solution of claim 36 in which said antithrombogenic agent is heparin.

38. The solution of claim 36 in which said viscosity is 10,000 to 50,000 cp.
